(19) United States
(12) Patent Application Publication
(10) Pub. No.: US 2007/0116773 A1
(43) Pub. Date: May 24, 2007

(54) Metal nanostructures and pharmaceutical compositions

(75) Inventors: Filip Frederix, Leuven (BE); Bieke Van de Broek, Genk (BE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Interuniversitair Microelektronica Centrum (IMEC), Leuven (BE)

(21) Appl. No.: 11/479,388

(22) Filed: Jun. 30, 2006

Related U.S. Application Data
(60) Provisional application No. 60/696,576, filed on Jul. 5, 2005.

(51) Int. Cl. A61K 9/14

(52) U.S. Cl. 424/489; 977/906

(57) ABSTRACT

A metal nanostructure is described. Such a metal nanostructure may comprise a nanometric metal core comprising gold, silver or an assembly or alloy of gold and silver, and one or more molecules attached to one or more surfaces of the nanometric metal core, where each of the molecules has the structural formula W-X-Y-Z, where W is an atom or a chemical group bound to the nanometric metal core, X is a hydrophobic spacer, Y is a hydrophilic spacer and Z is either hydrogen or a reactive group able to bind a reactive substrate or biomolecule. Such a metal nanostructure may be useful in making pharmaceutical compositions.

### PRIORITY

The present application claims priority to the following U.S. filed provisional patent application: U.S. 60/696,576, filed Jul. 5, 2005, which is incorporated herein by reference.

### FIELD

The present invention relates to metal nanostructures and methods of preparation thereof, and more particularly to metal nanostructures that have a stable shape and/or reactive groups on their surface, metal nanostructures that are linked to biomolecules, metal nanostructures that may be used in the preparation of pharmaceutical compositions, and films that comprise metal nanostructures.

### BACKGROUND

Shape has a strong influence on the physical properties of metal nanostructures. For example, gold nanostructures of different shapes behave differently in the presence of electromagnetic waves such as infrared (IR), visible, or ultra-violet (UV) irradiation. In sensing, plasmonic or hyperthermia applications, nanostructures having non-spherical shapes typically perform better than their spherical counterparts. In thermotherapy (i.e., heat treatment) the temperature of a tissue is artificially elevated with the aim of gaining therapeutic benefits. Thermotherapy is considered an adjunct to other treatments. For example, raising the temperature of tumors is one way to selectively destroy cancer cells. One problem with treating cancer successfully is the fact that cancerous cells are very difficult to target specifically. In most respects, they are like normal cells, and even if they are not, there differences may not be easily apparent. However, thermotherapy may be employed as malignant cells are reliably more sensitive to heat than normal cells.

Thermotherapy may exert its beneficial effect in several ways. Several studies have shown increased apoptosis in response to heat. Hyperthermia damages the membranes, cytoskeleton, and nucleus functions of malignant cells. It causes irreversible damage to cellular perspiration of these cells. Heat above 41° C. also pushes cancer cells toward acidosis (decreased cellular pH) which decreases the viability of the cells and their transplant ability. Tumors have a tortuous growth of vessels providing them blood, and these vessels are unable to dilate and dissipate heat as normal vessels do. Tumors, therefore, take longer to heat up. When tumors do heat up, they concentrate the heat within themselves. Tumor blood flow is increased by hyperthermia despite the fact that tumor-formed vessels do not expand in response to heat. Normal vessels are incorporated into the growing tumour mass and are able to dilate in response to heat, and to channel more blood into the tumor.

Tumor masses tend to have hypoxic (oxygen deprived) cells within the inner part of the tumor. These cells are resistant to radiation, but they are very sensitive to heat. This is why hyperthermia is an ideal companion to radiation: radiation kills the oxygenated outer cells, while hyperthermia acts on the inner low-oxygen cells, oxygenating them and so making them more susceptible to radiation damage. It is also thought that hyperthermia's induced accumulation of proteins inhibits the malignant cells from repairing the damage sustained.

All these properties make from thermotherapy a promising technique to combat cancer. Since the shape of metal nanostructures influences their response to infra-red radiation, shape control of metal nanostructures for use in thermotherapy is important.

The exact nucleation mechanism involved in the synthesis of metal nanostructures remains a mystery due to the length and the time scales on which it occurs and the variety of systems in which it may take place. Growth of the nucleus into larger nanocrystallites (i.e. seeds) likely occurs through a combination of aggregation and atomic addition.

Various strategies have been followed to control the shape of nanostructures. They either involve varying the type of seeding method employed or the type of inorganic ions introduced in the crystallization media, the use of templates, the use of light or the use of capping agents. Facet-selective capping agents promote the abundance of a particular shape by selectively interacting with a specific crystallographic facet via chemical adsorption.

Some of those methods have been proved successful and cubes, triangular nanoplates, pyramids, branched and rod-like nanostructures have been produced. Although those special morphologies are promising in a variety of applications, they are currently not applied in real technologies. One of the major reasons is the stability of these nanostructures.

For example, synthesis and optical properties of branched gold nanocrystals have been described that result in nanostructures that can be kept for several days in a refrigerator, although eventually a spectral shift toward shorter wavelengths takes place, indicative of nanostructure annealing. This is indicative that those nanostructures lose their branched morphology and relax into spherical nanostructures.

Aside from the stability problem, it is also desirable that nanostructures contain functional groups which allow the nanostructures to covalently or electrostatically bind to biomolecules, surfaces or other materials. For example, the functionalization of gold hollow nanocages with compounds of the following formula:
IgG-HN—CO—CH₂—CH₂—(O—CH₂—CH₂)₈—S—S—(CH₂—CH₂—O)₈—CH₂—CH₂—CO—NH—IgG, where IgG is an antibody (anti-mouse immunoglobulin G). The aim of this functionalization is to target cancer cells.

### SUMMARY

Although such approaches may introduce functionalities into a nanostructure, shape stability needs to be considered. There is, therefore, a need in the art for a method to stabilise the shape of nanostructures. In particular, there is a need in the art for a method which permits to simultaneously stabilize the shape of nanostructures and introduce functionalities on the surfaces of nanostructures.

An object of the present invention is to provide metal nanostructures, especially non-spherical metal nanostructures with an improved shape stability. Another object of the present invention is to provide a method, in particular a water-based method in order to prepare these metal nanostructures.

Generally speaking, as will be described below, the shape of nanometric metal cores can be stabilized by contacting nanometric metal cores with one or more molecules of the general formula W′—X—Y-Z or Z-Y—X—W—W—X—Y-Z, where W and W′ are atoms or chemical groups able to bind to the nanometric metal core, X is a hydrophobic spacer, Y is a hydrophilic spacer and Z is either hydrogen or a reactive group able to bind a reactive substrate or biomolecule.

In one example, a metal nanostructure comprises a nanometric metal core comprising gold, silver or an assembly or alloy of gold and silver, and one or more molecules attached to one or more surfaces of the nanometric metal core, wherein the molecules have the general formula W—X—Y-Z, where W is an atom or a chemical group which is bound to the nanometric metal core, X is a hydrophobic spacer, preferably a hydrocarbon chain having from 8 to 30 carbon atoms, Y is a hydrophilic spacer and Z is either hydrogen or a reactive group able to bind a reactive substrate or biomolecule, preferably covalently or electrostatically.

This example is advantageous because the hydrophilic spacer Y permits a water-based preparation of the metal nanostructure and improves the water-suspendability of the metal nanostructures while the hydrophobic spacer X ensures the stability of the W—X—Y-Z surface film itself.

As an additional feature, at least one of the one or more molecules may have a Z reactive group able to bind, preferably covalently or electrostatically, a reactive substrate or a biomolecule. As an additional sub-feature of the above described additional feature, the metal nanostructure may have one or more reactive group Z attached to a biomolecule. This feature is advantageous in medical applications because it permits to target the metal nanostructure to a specific location in a mammalian body for which the biomolecule presents a particular affinity.

As an additional feature, the nanometric metal core may have each of its dimensions from about 1 to 100 nm. This feature is advantageous in medical applications because its small size permits it to enter cancer cells and confer particular electromagnetic properties to the metal nanostructures. As an additional feature, the metal nanostructure may have a pyramidal or a branched shape. This feature is advantageous because such shapes permit the production of much heat under IR irradiation, when compared to metal nanostructures of spherical shape.

As an additional feature according to the present invention, one or more, preferably each, of the W—X—Y-Z molecules may form a monolayer at one or more surfaces of the nanometric metal core. This feature is advantageous, because it enables to cost-efficiently cover the whole surface of the nanometric metal core, thereby stabilizing it.

As an additional feature, W may be an atom selected from sulfur and selenium. This feature is advantageous because sulfur and selenium atoms have a high affinity for gold and/or silver and/or their alloys.

As an additional feature, Y may be a hydrophilic spacer that does not contain hydrogen bond donors but does contain hydrogen-bond acceptors and is overall electrically neutral. This feature is advantageous because these properties are favourable to avoid non-specific binding to biomolecules, cells or tissues.

As an additional feature, Y may be a polyethylene glycol, a monosaccharide, an oligosaccharide, a polysaccharide, a N-acetylpiperazine, a permethylated sorbitol group or an oligosarcosine. This feature is advantageous because in medical applications these hydrophilic linkers are biocompatible and have also the property to avoid non-specific binding to biomolecules, cells or tissues.

In another example, a pharmaceutical composition may include the metal nanostructures described above and optionally one or more pharmaceutically acceptable excipients. The proportion of metal nanostructures in this composition may be from about 0.01 to 99.99% by weight, preferably 0.1-10%. This is advantageous because the metal nanostructures of the present invention can be used in medical applications like the hypothermic treatment of cancer.

As an additional feature, this pharmaceutical composition is active under infrared radiation, preferably at a wavelength between about 750 and about 1400 nm, which is advantageous because at these wavelengths the metal nanostructures are the most responsive.

In another example, a process for preparing a metal nanostructure is described that is characterized in that a nanometric metal core is contacted with one or more molecules having the general formula W′—X—Y-Z or Z-Y—X—W—W—X—Y-Z, wherein W and W′ are atoms or chemical groups able to bind to the nanometric metal core, X is a hydrophobic spacer, Y is a hydrophilic spacer and Z is either hydrogen or a reactive group able to bind a reactive substrate or biomolecule.

As an additional feature, one or more capping agents may be attached to the nanometric metal core and the preparation of the metal nanostructures is then carried out by exchanging these capping agents with one or more molecules having the general formula W′—X—Y-Z or Z-Y—X—W—W—X—Y-Z. This feature is advantageous because it enables to stabilize the non-spherical shapes obtainable when the nanometric metal core is synthesized in presence of capping agents. In the absence of such capping agents, shape control may often be inefficient and/or very limited in extent.

As an additional feature, the one or more molecules having the general formula W′—X—Y-Z or Z-Y—X—W—W—X—Y-Z may be water soluble. This is advantageous because it permits the preparation method of the metal nanostructures of this invention to be water-based.

As an additional feature, the capping agents may be selected from CH₃ (CH₂)ₙN⁺(CH₃)₃X⁻(wherein n=integer of 1 to 15 and X=Br or Cl), (CH₃(CH₂)ₙ)₄N^{+X}⁻(wherein n=integer of 1 to 15 and X=Br or Cl), benzyldimethylammonium chloride (BDAC), bis(p-sulfonatophenyl)phenylphosphine (BSPP), bis(2-ethylhexyl)sulfosuccinate (AOT), octylamine, dimyristoyl-L-alpha-phosphatidyl-DL-glycerol (DMPG), sodium dodecylsulfonate (SDS), ascorbic acid, and sodium citrate. This is advantageous because these capping agents are readily available and known to efficiently induce specific shapes to metal nanostructures containing gold or silver.

As an additional feature, W′ may be a chemical group selected from R—S— and R—Se— and W may be an atom selected from sulfur and selenium, wherein R is selected from hydrogen and C₁-C₃₀ alkyl chains.

In another example, a film may comprise the metal nanostructures, where a process for making the includes suspending metal nanostructures in an aqueous medium and depositing the resulting suspension onto a substrate.

In another example, metal nanostructures may be used to measure a change of refractive index. More specifically, a change of refractive index may be measured by forming a film that comprises metal nanostructures, measuring the wavelength of maximal absorption of the film in a medium having a first refractive index, measuring the wavelength of maximal absorption of the film in a medium having a second refractive index, calculating the difference between the wavelength of maximal absorption measured in the two films, and relating this calculated difference to a change in refractive index.

In another example, a method of therapy using metal nanostructures may be applied to a patient, preferably a mammal such as a human being, having cancer tumor, comprising the steps of injecting metal nanostructures into a tumor and exposing the tumor to infrared irradiation, preferably at a wavelength between about 750 and about 1400 nm.

As an alternative example, the method of therapy comprises the step of injecting into the bloodstream of the patient a metal nanostructure attached to a biomolecule, where the biomolecule has a particular affinity for cancer cells, and exposing the tumor to infrared irradiation.

These as well as other aspects and advantages will become apparent to those of ordinary skill in the art by reading the following detailed description, with reference where appropriate to the accompanying drawings. Further, it is understood that this summary is merely an example and is not intended to limit the scope of the claims.

### FIGURES

Certain examples are described below in conjunction with the appended drawing figures, wherein like reference numerals refer to like elements in the various figures, and wherein:

FIG. 1 is a schematic representation of a process for preparing metal nanostructures, according to an example;

FIG. 2 is a schematic representation of a process for reacting biomolecules to nanometric metal cores, according to an example;

FIG. 3 is a graph that shows the dependence of the optical properties of branched nanometric gold cores toward the addition rate of HAuCl₄, according to an example;

FIG. 4 is a graph that shows the influence of the age of the mixture citrate/BSPP on the band positions of absorption maxima of branched nanometric gold cores, according to an example;

FIG. 5 is a graph that shows the influence of the HAuCl₄ concentration on the band positions of absorption maxima of branched nanometric gold cores, according to an example;

FIG. 6 is a graph shows the influence of the amount of BSPP on the band positions of absorption maxima and band width of branched nanometric gold cores, according to an example;

FIG. 7 is a graph shows the influence of the amount of H₂O₂ on the band positions of absorption maxima and band width of branched nanometric gold cores, according to an example;

FIG. 8 is a graph that shows the UV-Visible spectra of branched nanometric gold cores plated with various amounts of silver in solution, according to an example;

FIG. 9 is a graph that shows the UV-Visible spectra of branched nanometric gold cores plated with silver on a surface, according to an example;

FIG. 10 is a graph that shows the UV-Visible spectra of branched metal nanostructures, according to an example;

FIG. 11 is a graph that shows the FTIR spectrum of metal nanostructures, according to an example;

FIG. 12 is a graph that shows the UV-Visible spectra of branched metal nanostructures coupled to IgG, according to an example;

FIG. 13 is a graph that shows the UV-Visible spectra of branched metal nanostructures, according to an example;

FIG. 14 is a graph that shows the FTIR spectrum of metal nanostructures, according to an example;

FIG. 15 is a graph that shows an absorption spectrum showing the improved stability of metal nanostructures, according to an example; and

FIG. 16 is a graph that shows an absorption spectra showing the sensibility of metal nanostructure films toward the refractive index of the surrounding media, according to an example.

### DESCRIPTION

The present invention relates to metal nanostructures. In one example, these metal nanostructures are composed of a nanometric metal core having organic molecules linked at the surface thereof. Preferably, these molecules form a monolayer at the surface of the nanometric metal core. The nanometric metal core comprises gold and/or silver which can be optionally assembled together, alloyed or form an assembly or alloy with one or more other metals in proportions well known to the skilled person.

The nanometric metal cores are preferably particles having each dimension from 1 to 100 nm. Preferably, at least one dimension of said particles is from 10 to 90 nm, more preferably 20 and 75 nm. The metal nanostructures can have any shape such as, but not limited to, a sphere shape, a rod shape, a disk shape, a branched shape, a cuboidal shape or a pyramidal shape. Branched nanostructures are nanoparticles having one or more tips extending toward the outside of the particle.

The molecules attached to the nanometric metal core have the general formula W—X—Y-Z, wherein W is an atom or a chemical group which is bound to the nanometric metal core, X is a hydrophobic spacer, Y is a hydrophilic spacer and Z is either hydrogen or a reactive group. Suitable X hydrophobic spacers include, but are not limited to, hydrocarbon chains of the formula —CH₂)ₙ-wherein n is from 8 to 30, preferably 9 and 30. X is important to realize stable self-assembled mono-layers and therefore stable metal nanostructures. Preferably, at least one molecule linked to the surface of the nanometric metal core has Z being a reactive group. Alternatively, all molecules present at the surface of the nanometric metal core have a Z being a reactive group. Suitable atoms or chemical groups that W may comprise, but are not limited to, are atoms such as sulfur or selenium.

Suitable Y hydrophilic spacers are spacers preferably allowing solubility of the molecules in water-based solutions. Preferably, the Y spacer also avoids non-specific adsorption to biological compounds, cells or tissues. For this purpose, Y should preferably contain hydrogen-bond acceptors (e.g. fluorine, oxygen or nitrogen atoms possessing a lone electron pair), should preferably not contain hydrogen bond donors (atoms of fluorine, oxygen or nitrogen covalently bond to a hydrogen atom), and should preferably be overall electrically neutral. Preferably, the Y hydrophilic spacer comprises polyethylene glycol, monosaccharides, oligosaccharides, polysaccharides, N-acetylpiperazine oligo(sarcosine) or permetylated sorbitol. Alternatively, the Y spacer can be made of two parts: for example, a first part increasing the solubility in water (e.g. an ether, a crown ether, an amine, an amonium salt, an amide, an amino acid, a peptide, a sugar, a nitrile, a phosphonium salt or a phosphate) and a second part avoiding non-specific adsorption (e.g. polyethylene glycol). Due to the hydrophobic nature of the X compound (which may be necessary for stability), Y may be necessary to allow water-solubility of the molecules.

The reactive group Z may be any reactive group able to form a covalent bond with a reactive substrate or biomolecule. According to an embodiment of the present invention, Z is preferably but not limited to one of the common functional groups, including amines, thiols, carboxylic acids, and alcohols and can also be selected from one of the following species:

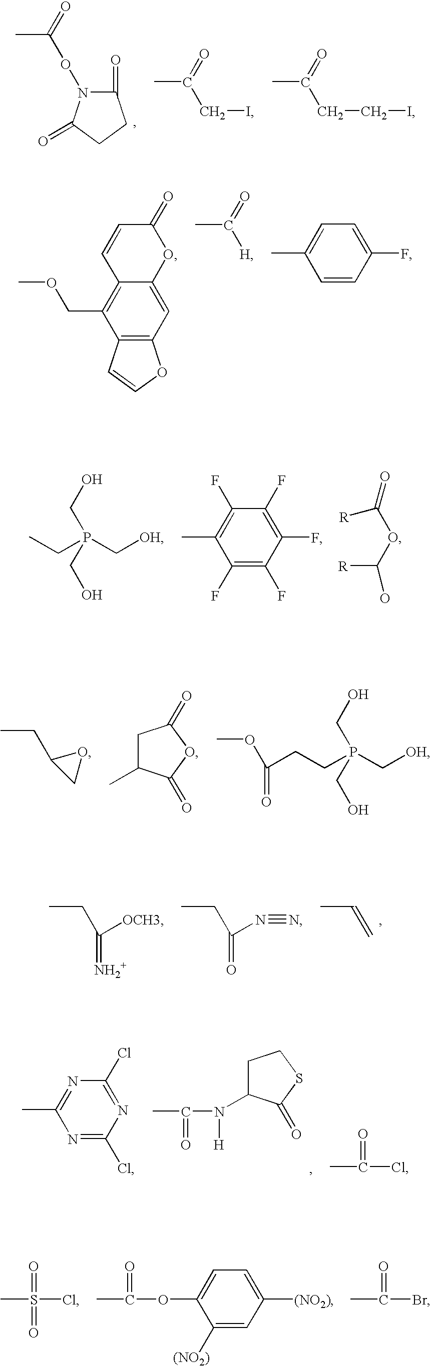

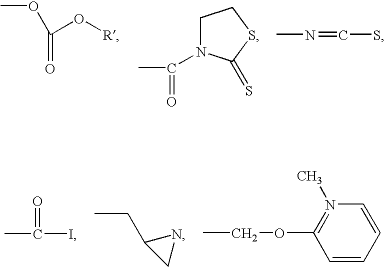

The metal nanostructures are preferably water-suspendible under normal conditions. Their water suspendibility is conferred by the Y spacers.

In another example, a process for preparing the metal nanostructures is described below. This process is performed by contacting a nanometric metal core such as defined above with one or more organic molecules having the general formula W′—X—Y-Z or Z-Y—X—W—W—X—Y-Z. By this process, a self-assembled monolayer of these organic molecules is formed around the nanometric metal core and the particular morphology of the nanometric metal core is not changed. Additionally, the metal nanostructures are stabilized by this process. W, X, Y and Z are as defined above and W′ is an atom or a chemical group able to bind the nanometric metal cores. Preferably, W′ is a chemical group selected from thiol, selenol, sulfide of the general formula —S—R and selenide of the general formula —Se—R wherein R is either a hydrogen or a C₁-C₃₀ alkyl chain, for example. Suitable X hydrophobic spacers comprise but are not limited to hydrocarbon chains having from 8 to 30 carbon atoms, preferably from 9 to 30 carbon atoms.

As another example, the nanometric metal cores are preferably produced in such a way as to obtain nanometric metal cores of controlled shape. In this example, the nanometric metal cores are grown in the presence of one or more capping agents. Typical capping agents include, but are not limited to, surfactants. Examples of suitable capping agents include, but are not limited to surfactants of the general formula CH₃(CH₂)ₙN⁺(CH₃)₃X⁻(n=integer of 1 to 15 and X=Br or Cl) (e.g. hexadecyltrimethylammonium bromide (CTAB)) or of the general formula (CH₃(CH₂)ₙ)₄N⁺X⁻(n=integer of 1 to 15 and X=Br or Cl), benzyldimethylammonium chloride (BDAC), bis(p-sulfonatophenyl)phenylphosphine (BSPP), bis(2-ethylhexyl)sulfosuccinate (AOT), octylamine, dimyristoyl-L-alpha-phosphatidyl-DL-glycerol (DMPG), sodium dodecylsulfonate (SDS), ascorbic acid, sodium citrate and the like. In a preferred embodiment, the process is water-based where the nanoparticles are suspended in an aqueous solution. Another process for preparing the metal nanostructures of the present invention consists in contacting nanometric metal cores capped with capping agents as defined above with one or more molecules having the general formula W′—X—Y-Z or Z-Y—X—W—W—X—Y-Z as defined above and thereby exchanging the capping agents for the one or more molecules of the general formula W—X—Y-Z. as defined above.

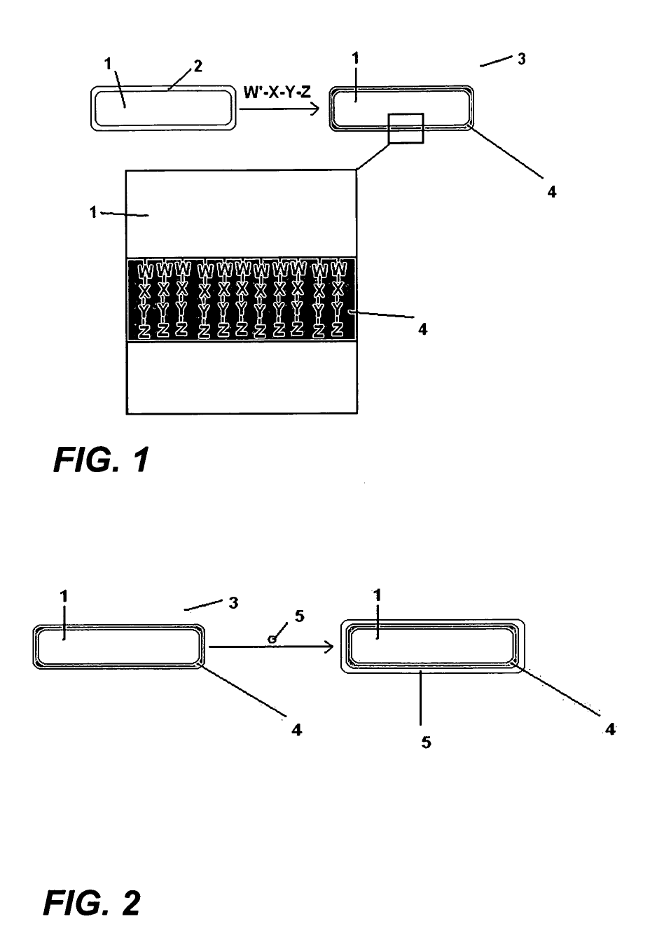

FIG. 1 schematically shows the transformation of a nanometric metal core (1) surrounded by capping agents (2) into a metal nanostructure (3) via the exchange of the capping agent (2) by a self assembled monolayer (4) of molecules having the general formula W—X—Y-Z.

In yet another example, the metal nanostructures are linked to one or more biomolecules via reaction or interaction of at least one reactive group Z with said one or more biomolecules. In a preferred but non-limiting example, the biomolecules may be anti-bodies, antibody fragments or other receptors targeting cancer cells. This is schematically represented in FIG. 2 showing the attachment of the presently contemplated nanostructures (3) to biomolecules (5).

In another example, the metal nanostructures of the present invention may be used to treat cancer by thermotherapy. The metal nanostructures of the present invention absorb near infra-red (hereinafter referred as NIR) light, i.e. light with a wavelength comprised between about 750 and about 1400 nm. Upon irradiation, the presently contemplated metal nanostructures generate heat which can be used to kill cancer cells. Ideally the metal nanostructures should:
be sufficiently small to penetrate through the cancer cells,
produce enough heat to kill the cells,
be functionalized with antibodies, antibody fragments or other biomolecules targeting cancer cells, and
be stable upon heating and in function of time.

All these desired properties may be achieved using the presently contemplated metal nanostructures.

In a first step, the method of treatment is carried out by either injecting the metal nanostructures directly into the tumor or in the bloodstream of the patient having a cancer tumor. In the latter case, the metal nanostructure should be linked to a biomolecule targeting cancer cells as described above. In a second step, the metal nanostructures now present in the tumor region are subjected to infrared irradiation. The resulting local increase in temperature causes the death of the tumor cells. In another example, the presently contemplated metal nanostructures are included in a pharmaceutical composition or are used in the manufacture of a pharmaceutical composition. This pharmaceutical composition is active against cancer under infrared irradiation.

Another example relates to a film consisting of metal nanostructures. In such an example, the film can be obtained by coating a substrate with a suspension of the presently contemplated metal nanostructures. The method of coating can be any method known in the art such as but not limited to spraying, curtain coating, roller coating, doctor blade coating, dip coating, spin-coating, screen printing, etc. The substrate can be any substrate but if the film is meant to be used in localized surface plasmon resonance sensors, the substrate is preferably transparent such as but not limited to PMMA, PS, glass or quartz. Quartz is the most preferred substrate. Preferably, the substrate is silanized in order to improve adherence of the film on the substrate. Metal nanostructures change their absorption behavior upon differences in the refractive index at their surfaces. This property can be applied to sensing binding events like in the case of localized surface plasmon resonance sensors.

An illustration of how the film of the present invention can be used in a localized surface plasmon resonance sensor is given in example 4 below.

The presently contemplated nanostructures may also be used in sandwich assays. A sandwich assay is the base of one of the currently most applied diagnostic methods, namely the ELISA test. In such an assay, an antibody probe is firstly immobilized onto a solid support, next this antibody probe can bind the antigen target. In a classical ELISA this binding event is visualised by contacting this complex with a secondary antibody which contains a certain label. These labels are typically absorption molecules, fluorescence labels or enzymatic labels. However, sometimes also metal nanostructures (e.g. Au or Ag) can be used to enhance the signal or can be used in combination with (optical) biosensor techniques. The extraordinary optical properties of non-spherical nanostructures, make them ideal as optical labels. However, the stability and the functionalization are key issues to use these nanostructures for this sort of applications. Due to their stability, the metal nanostructures of the present invention are therefore good candidates for use in sandwich arrays.

Various examples are now described below, relating to howWe now discuss in some details how nanometric metal cores of specific shape and composition, capped with capping agents, can be synthesized. Next, also, working examples are given as to how such nanometric metal cores of specific shape and compositions, capped with capping agents, can be stabilized by exchanging these capping agents with one or more molecules of the general formula W—X—Y-Z.

Branched Nanometric Gold Cores

The synthesis of branched nanometric gold cores may be based on a procedure where sodium citrate and bis(p-sulfonatophenyl)phenylphosphirie dihydrate dipotassium (BSPP) are mixed and H₂O₂ is added. Next, under constant shaking 0.05 M HAuCl₄ is added slowly at room temperature. Over several minutes, the solution color changed from colorless to blue. The resulting blue suspension can be kept in the refrigerator for several days, although eventually a spectral shift to the blue takes place. The nanocrystals lose their branched morphology and relax into spherical particles. The amount and the sharpness of these branches is influenced by several parameters: the addition rate of HAuCl₄, the age of the mixture of citrate and BSPP, the concentration of HAuCl₄, the amount of BSPP, and the amount of H₂O₂.

Addition Rate of HAuCl₄

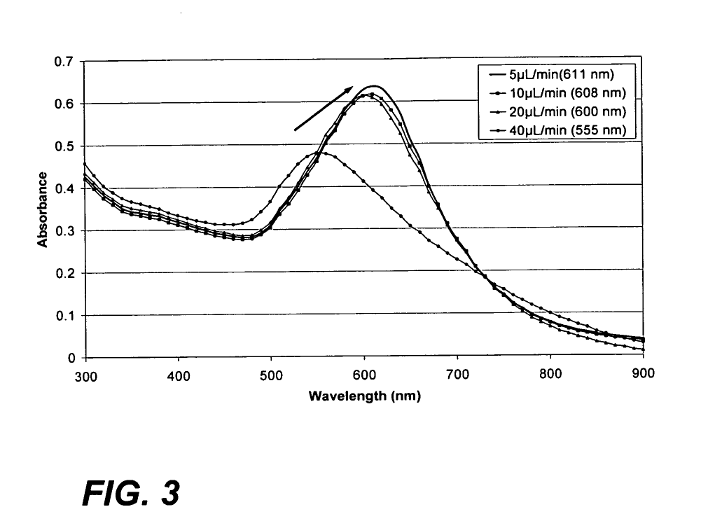

HAuCl₄ is the precursor salt that is reduced to form colloidal gold. This salt must be added slowly so the branches can grow in certain directions. In the example of FIG. 3, the addition rate was varied from 5 to 40 μL per minute. The slower HAuCl₄ is added, the more red-shifted the absorption maximum (FIG. 3).

FIG. 3 shows the UV-Vis absorption spectrum of the addition rate of HAuCl₄. The solution color changes from red to purple to blue when going from faster to slower addition rate respectively.

Age of the Mixture of Citrate and BSPP

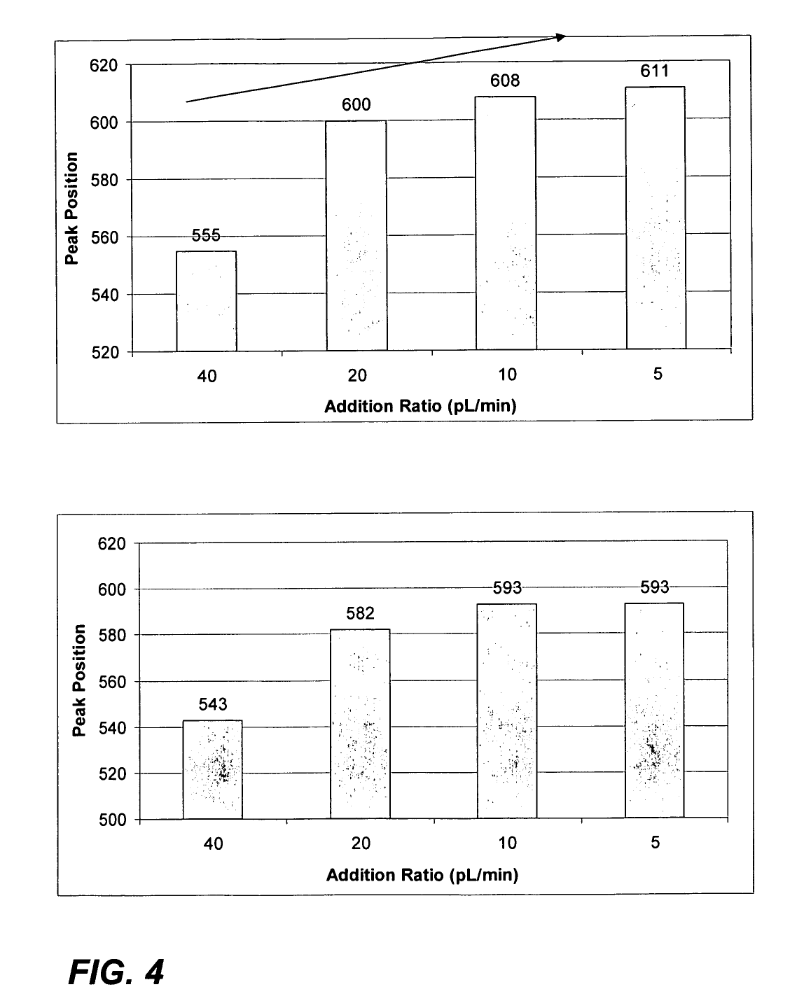

Citrate is a reducing agent for the reduction of HAuCl₄ into colloidal gold. It is also the capping agent that stabilizes the nanometric metal core in the beginning. BSPP is a detergent necessary to induce the growth in certain directions resulting in the branched morphology. Unfortunately, the shape only remains stable for a few days. Then the nanometric metal cores relax into a spherical morphology. This mixture of citrate and BSPP should be made freshly. In this way the branched nanometric metal cores show the best characteristics: the UV-Vis absorption maximum was shifted to higher wavelengths. When using a mixture that was one week old, the UV-Vis absorption maximum is shifted to lower wavelengths (see FIG. 4).

FIG. 4 compares band positions of absorption maxima of branched nanostructures synthesized with a freshly prepared mixture (left) and synthesized with a mixture of one week old (right).

The Concentration of HAuCl₄

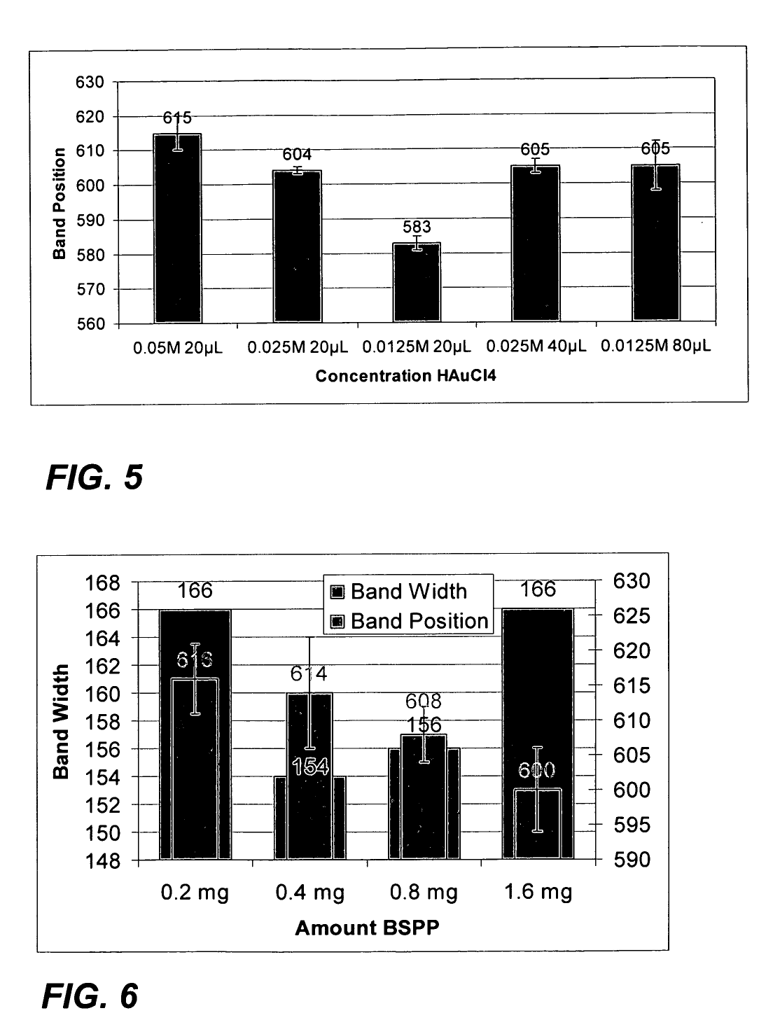

The concentration of HAuCl₄ was varied from 0.05M to 0.0125M for an addition rate of 10 μL/min. Firstly, the same amount (20 μL) of HAuCl₄ for the different concentrations was taken. Secondly, the amount (40-80 μL) was increased when decreasing the concentration (0.025-0.0125M). The best results were obtained with 20 μL of 0.05M HAuCl4 (see FIG. 5).

FIG. 5 shows the band positions of absorption maxima for branched nanostructures synthesized with different concentrations of HAuCl₄.

The Amount of BSPP

BSPP is a detergent that needs to be used to induce the growth in certain directions resulting in the branched morphology. The amount is varied from 0.2 to 1.6 mg in 10 mL of citrate solution. The best result concerning the position of the absorption maximum was obtained with 0.2 mg of BSPP (see FIG. 6).

FIG. 6 shows the band positions of absorption maxima and band widths for branched nanometric metal cores synthesized with different amounts of BSPP.

The Amount of H₂O₂

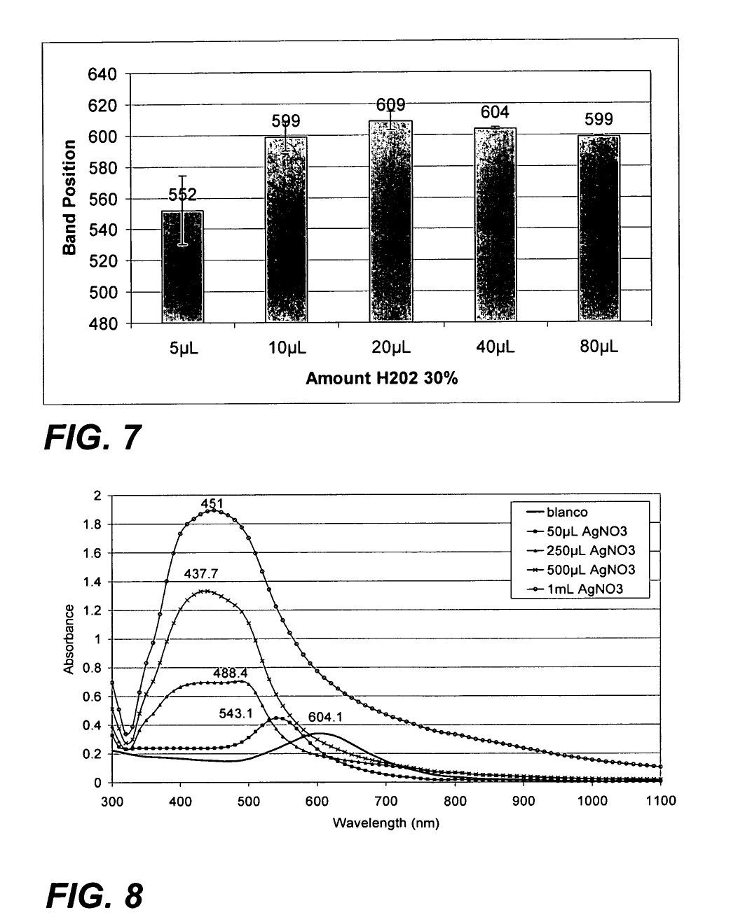

H₂O₂ is a reducing agent that is able to reduce HAuCl₄ together with citrate at room temperature. The amount of H₂O₂ was varied from 5 to 80 μL in 10 mL of citrate/BSPP solution. The best results were become with 20 μL of H₂O₂ (see FIG. 7).

FIG. 7 shows band positions of absorption maxima of branched nanometric metal cores synthesized with different amounts of H₂O₂.

Branched Nanometric Gold Cores Plated with Silver

Branched nanometric gold cores were plated with silver in solution. In a procedure, 2.5 mL of branched nanometric gold cores were mixed with 1.5 mL of Ascorbic Acid 10⁻² M (reducing agent). Then 20 mL of H₂O was added under constant shaking. Finally, different amounts of AgNO₃ 10⁻² M were added going from 0 μL (blanco) to 1 mL of AgNO₃. Upon addition of AgNO₃ the absorption maximum of the gold shifts to lower wavelength and disappears slowly when a new absorption maximum of silver appears (see FIG. 8).

FIG. 8 shows the UV-Vis absorption spectra of branched nanometric gold cores plated with silver in solution.

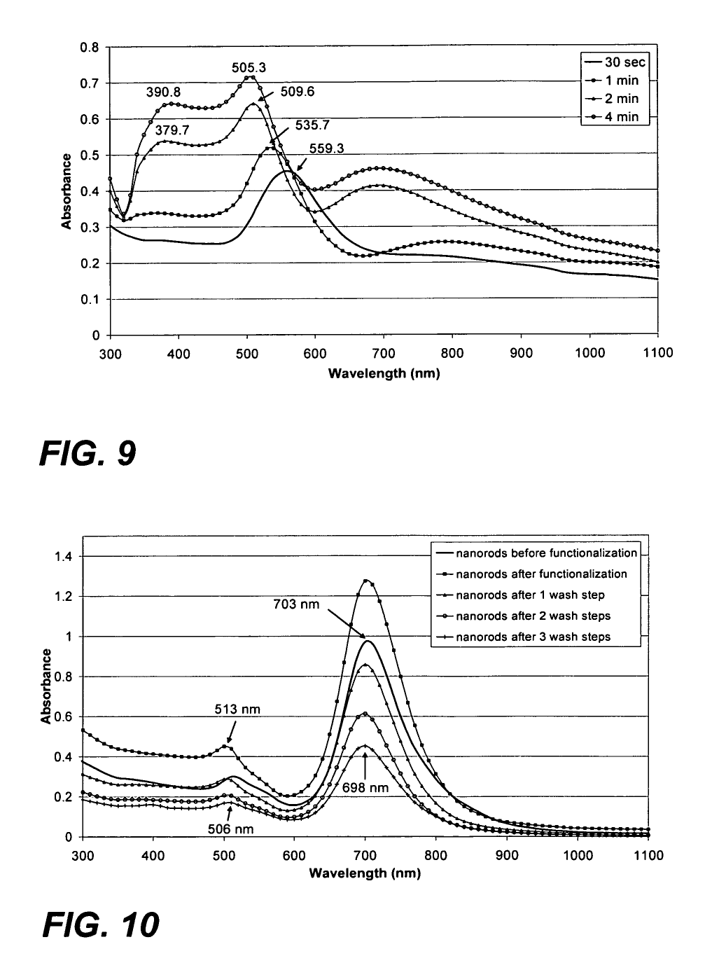

Branched nanometric gold cores were also plated with silver on a surface. The branched nanometric gold cores were deposited on a silanized quartz substrate. Then these substrates were held in a 1/1 mixture of AgNO₃ and hydroquinone for different periods of time going from 30 seconds to 4 minutes. For short periods of time the absorption maximum shifts to lower wavelengths. For longer plating times (2 and 4 minutes) a second absorption maximum appears around 400 nm because of the larger amount of plated silver (see FIG. 9).

FIG. 9 shows the UV-Vis absorption spectra of branched nanometric gold cores plated with silver on a surface.

Gold Nanorods

Others have made nanocrystals with other morphologies like cubes, triangular nanoplats, pyramids, etc. One morphology of particular interest may be nanorods. Each of these morphologies start with the synthesis of little gold nanoparticles (seeds) which then grow into nanorods in a second step.

### EXAMPLE 1

### Preparation of a Stabilized Gold Nanorods Solution

0.250 ml of an aqueous 0.01 M solution of HAuCl₄.3H₂O was added to 7.5 ml of a 0.10 M CTAB solution in a test tube. The solution was gently mixed by inverting the tube repeatedly. The solution appeared bright brown-yellow in color. Then, 0.600 ml of an aqueous 0.01 M ice-cold NaBH₄ solution was added at once, followed by a rapid mixing (by inverting the tube) for 2 min. Care was taken to allow the escape of the evolved gas during mixing. The solution developed a pale brown-yellow color.

2 mL of the gold nanorods containing solution was brought to pH=11 with NaOH. Next, the gold nanorods were mixed with 200 μL of 12 mM of S₂—C₁₁—PEO₄—CHO in water, which is an abbreviation for:

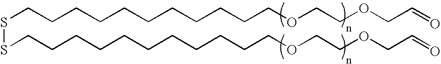

The function of this compound in to replace the original CTAB capping agents around the nanometric metal cores. This approach generated nanorods with aldehyde group functionalities. The aldehyde group functionalized gold nanorods were purified from the excess of S₂—C₁₁—PEO₄—CHO by ultracentrifugation of the gold nanorods. They were consequently washed with ultra-pure water. This procedure was repeated three times to ensure purified aldehyde group functionalised gold nanorods. In every purification step, some gold nanorods were lost but characteristic spectral properties of the gold nanorods were still visible. An alternative approach to purify the obtained nanostructures consists in performing a dialysis against ultrapure water.

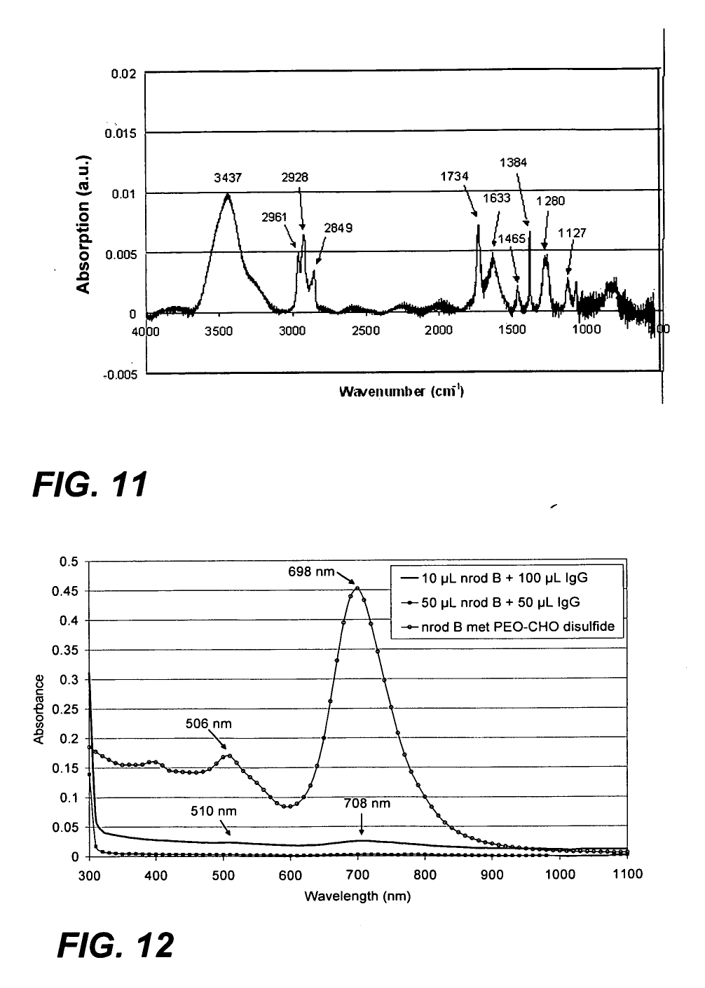

Experimental evidence that the functionalization was successful is shown in the UV-V is absorption spectrum (see FIG. 10) and in the Fourier Transformed infra Red (FTIR) spectrum (in a KBr pellet) of the resulting gold nanorods (see FIG. 11).

FIG. 10 shows the UV-visible absorption spectrum of gold nanorods before (plain line) and after the functionalization with S2—C₁₁—PEO₄—CHO molecules (squares) and after a first (triangles), a second (circles) and a third washing step (crosses). A shift in the peak position lo from 703 nm to 698 nm is a first indication that the functionalization was successful.

The attributions of the bands visible in FIG. 10 are detailed in Table 1 below.

TABLE 1

| Band (cm⁻¹) | Description |
|---|---|
| 3437 | Bounded H₂O |
| 2961 | (CH₂) near aldehyde |
| 2928 and 2849 | asymm and symm CH₂ |
| 1734 | (C═O) |
| 1633 | (OH) of H₂O |
| 1465 and 1384 | asymm and symm (CH₂) |
| 1280 and 1127 | asymm and symm |
| | (C—O—C) |

The morphology of the gold nanorods did not change during this functionalization step, which was verified by comparing TEM images before and after functionalization and purification of the gold nanorods.

The stability of these metal nanostructures was excellent over a long period of time (the shape and the spectral properties of these gold nanorods remained unchanged in months), which shows the strength and advantages of this approach.

### EXAMPLE 2

### Coupling of a Gold Nanorod with a Biomolecule

The functionalized gold nanorods of example 1 were covalently coupled to antibodies. For this purpose, 10 μL of the functionalized gold nanorods solution was mixed with 100 μL of 2 mg/mL IgG. This IgG solution was prepared by mixing 50 μL IgG of 6 mg/mL with 100 μL cyanoborohydride buffer (which was brought to pH=5). After coupling of the IgG to the functionalized gold nanorods, the nanorods were purified via ultracentrifuge and washed with phosphate buffer. The existence of the coupling was verified by the observation of a shift from 698 nm to 708 nm in the peak position in the resulting absorption spectrum (see FIG. 12).

FIG. 12 shows the UV-visible spectrum of the functionalized gold nanorods before (circles) and after IgG coupling by using a ratio nanorod: IgG of 1:10 (plain line) or 1:1 (squares).

### EXAMPLE 3

### Preparation of Stabilised Branched Gold Nanostructures

100 ml of a 6.8×10-3 M sodium citrate solution and 4 mg bis (p-sulfonatophenyl)phenylphosphine dihydrate dipotassium (BSPP) were mixed and 0.2 ml of 30% H₂O₂ was added. Next, under constant shaking 200 μl of 0.05M HAuCl₄ was added slowly at room temperature. Over several minutes, the solution colour changed from colourless to blue.

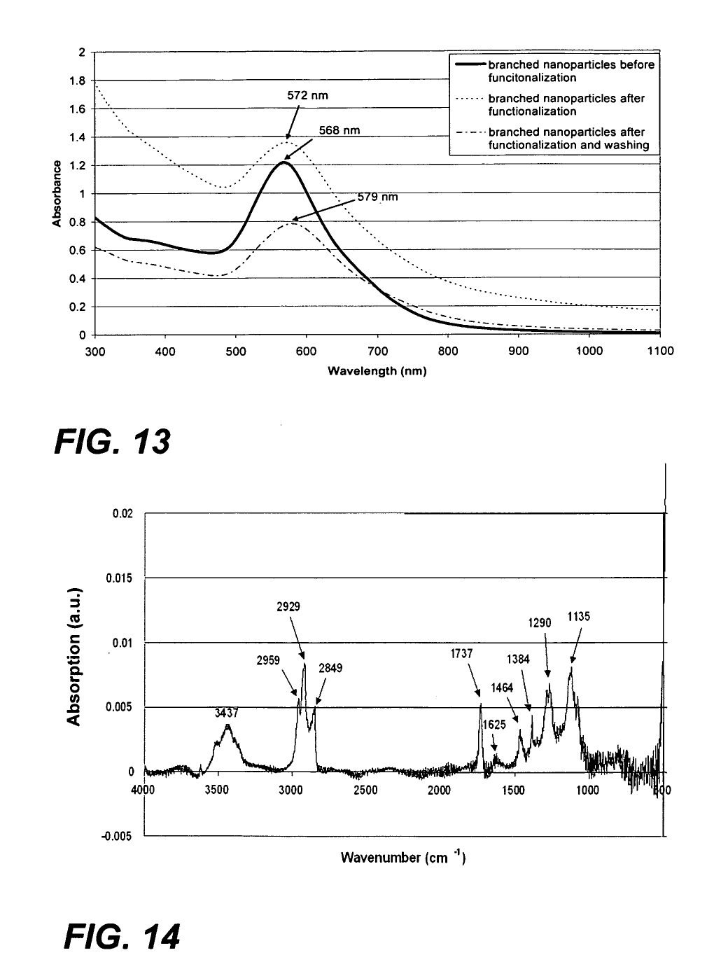

2 mL of this solution was brought to pH=11 with NaOH. Next, the nanorods were mixed with 200 μL of 12 mM of S₂—C₁₁—PEO₄—CHO in water. The purpose of this compound is to replace the original BSPP/citrate capping around the nanometric metal cores. This approach generates branched metal nanostructures with aldehyde group functionality. The aldehyde group functionalized branched metal nanostructures are purified from the excess of S₂—C₁₁—PEO₄—CHO by ultracentrifugation of the branched metal nanostructures to the bottom of the vial and wash them consequently with ultra-pure water. This procedure was repeated three times to ensure purified aldehyde group functionalized branched metal nanostructures. By every purification step, some branched metal nanostructures are lost but characteristic spectral properties of the branched metal nanostructures are still visible (see FIG. 13). An alternative approach to purify the obtained metal nanostructures consists in performing a dialysis against ultra-pure water.

FIG. 13 shows the UV-visible absorption spectrum of branched nanostructures before (plain line), after the functionalization with S₂—C₁₁—PEO₄—CHO molecules (dotted line), and after the purification of these branched metal nanostructures (dash-dot line). A shift in the peak position from 568 nm to red-shifted wavelength is a first indication that the functionalization was successful.

An experimental evidence that the functionalization was successful is shown in the Fourier Transformed infra Red (FTIR) spectrum (in a KBr pellet) of the resulting gold nanorods (see FIG. 14).

The morphology of the branched metal nanostructures did not change during this functionalization step, which was verified by comparing TEM images before and after functionalization and purification of the gold nanorods.

The stability of these metal nanostructures was excellent over a long period of time (months), which shows the strength and advantages of this approach. Not coated nanometric metal cores changed to spherical nanometric metal cores in a period of 2 to 3 days.

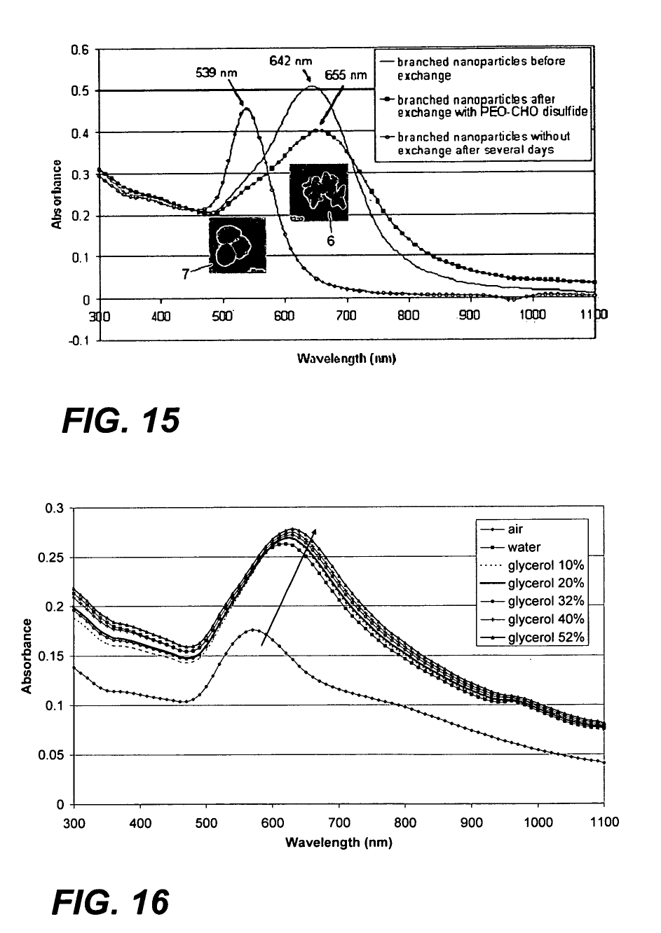

FIG. 15 shows the UV-Visible spectrum of branched nanometric gold cores before (plain line) and 3 days after exchange (squares) with S₂—C₁₁—PEO₄—CHO. Insert (6) in FIG. 15 shows a TEM picture of a gold nanostructure corresponding to the square line. It is clear from this picture that the gold nanostructures still kept their branched shape 3 days after synthesis and functionalization. The absorption spectrum of unfunctionalized branched nanometric gold cores 3 days after synthesis are presented for comparison purpose (circles). Insert (7) in FIG. 15 shows a TEM picture of a nanometric gold core corresponding to the circle line. It is clear from this picture that, contrary to the functionalized gold nanostructure, the unfunctionalized nanometric gold core came back to a spherical shape after three days. This change is accompanied by a large (>100 nm) blue shift.

### EXAMPLE 4

### Sensitivity of a Gold Nanostructure Film to Dielectric Environment

Metal nanostructure films (the metal used being specified in table 2 below) were obtained by deposition of metal nanostructures onto a silanized quartz substrate using metal nanostructures suspensions. Sensitivity measurements on these films were done by changing the dielectric environment of the metal nanostructures (different glycerol concentrations). Both the maximum absorbance and the wavelength at that position were monitored during variation of the refractive index (from 1.008 for air to 1.4011 for 52% glycerol).

FIG. 16 shows the UV-Vis absoption spectrum of the metal nanostructure film. It shows the red-shift in wavelength and the increase in absorbance observed for increasing concentration of glycerol.

The relative shift in peak position, Δλmax, and the relative shift in absorbance, Δabs, are linearly dependent on the refractive index of the surrounding medium. From the slope of these plots, which could be obtained by linear regression, the sensitivity of the films (Δλmax/Δn or Δabs/Δn) was calculated.

Table 2 below provides the slopes for the wavelength and absorbance curves.

TABLE 2

| Metal nanostructure film | Δλmax/Δn |
|---|---|
| Spherical gold (50 nm) | 64.79 |
| Branched gold | 144.02 |
| Branched gold + silver plating (on surface) | 197.09 (2 minutes plating) |
| Branched gold + silver plating (in solution) | 214.1 (250 μL AgNO3) |

Branched gold nanostructures with silver plating films give the highest sensitivity for changes in wavelength due to the change of refractive index. The spherical nanometric gold cores film give the lowest sensitivity for changes in wavelength due to change in refractive index.

We claim:

1. A metal nanostructure comprising:
a nanometric metal core comprising at least one of gold, silver, and at least one of an assembly and an alloy that each comprise gold and silver; and
at least one molecule attached to at least one surface of the nanometric metal core, wherein each of the at least one molecules has the structural formula W—X—Y-Z, and wherein W includes at least one of an atom and a chemical group bound to the nanometric metal core, X includes a hydrophobic spacer, Y includes a hydrophilic spacer, and Z includes at least one of a hydrogen and a reactive group that is able to bind to at least one of a reactive substrate and a biomolecule.

2. The metal nanostructure as in claim 1, wherein each dimension of the nanometric metal core is from about 1 to about 100 nm.

3. The metal nanostructure as in claim 1, wherein the metal nanostructure comprises at least one of a pyramidal and a branched shape.

4. The metal nanostructure as in claim 1, wherein the at least one molecule forms a monolayer at the least one surface of the nanometric metal core.

5. The metal nanostructure as in claim 1, wherein W comprises an atom selected from the group consisting of sulfur and selenium.

6. The metal nanostructure as in claim 1, wherein X comprises a hydrocarbon chain having from 8 to 30 carbon atoms.

7. The metal nanostructure as in claim 1, wherein Y comprises an electrically neutral hydrophilic spacer including hydrogen-bond acceptors that are free from hydrogen bond donors.

8. The metal nanostructure as in claim 1, wherein Y is selected from the group consisting of a polyethylene glycol, a monosaccharide, an oligosaccharide, a polysaccharide, a N-acetylpiperazine, an oligo(sarcosine), and a permethylated sorbitol group.

9. The metal nanostructure as in claim 1, wherein Z is selected from the group consisting of amines, thiols, carboxylic acids, and alcohols.

10. The metal nanostructure as in claim 1, wherein Z is selected from the group consisting of:

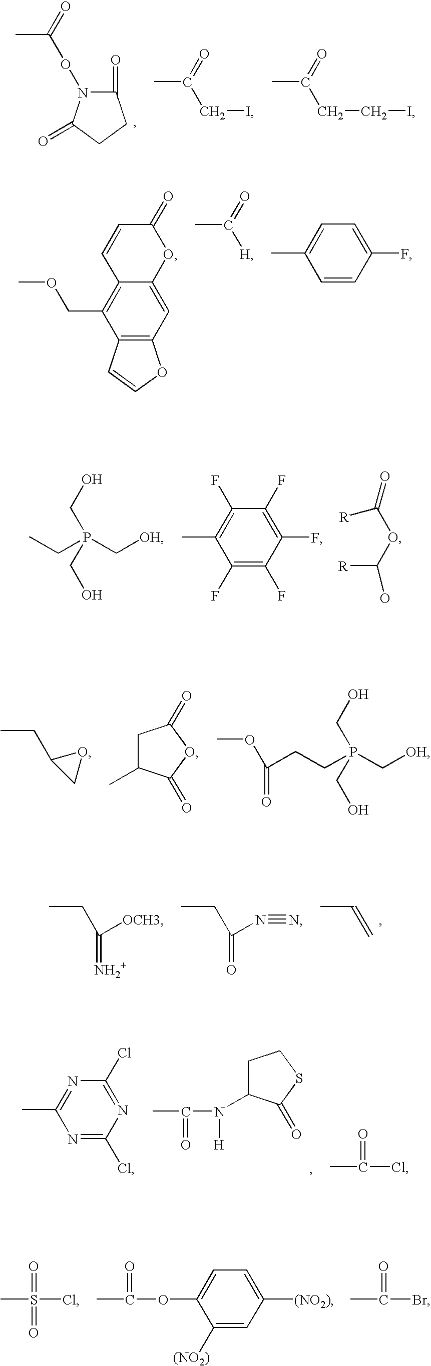

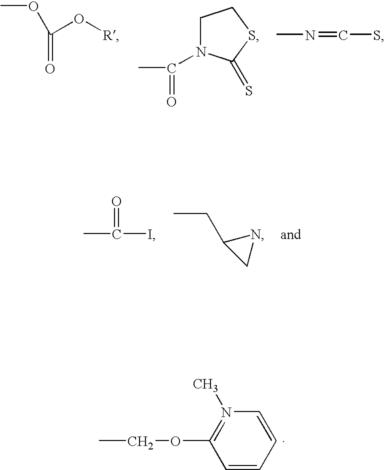

11. A process for preparing a metal nanostructure, wherein the process comprises contacting a nanometric metal core with at least one molecule having a structural formula of at least one of W′—X—Y-Z and Z-Y—X—W—W—X—Y-Z, wherein each of W and W′ comprises at least one of an atom or a chemical group able to bind to the nanometric metal core, X comprises a hydrophobic spacer, Y comprises a hydrophilic spacer, and Z comprises at least one of a hydrogen and a reactive group able to bind to at least one of a reactive substrate and a biomolecule.

12. The process as in claim 11, wherein at least one capping agent is attached to the nanometric metal core, and wherein the process further comprises exchanging the at least one capping agent with the at least one molecule.

13. The process as in claim 12, wherein the at least one capping agent is selected from the group consisting of CH₃(CH₂)ₙN+(CH₃)₃X⁻ and (CH₃(CH₂)ₙ)₄N⁺X⁻, wherein n=integer of 1 to 15 and X is slected from the group consisting of Br, Cl, benzyldimethylammonium chloride, bis(p-sulfonatophenyl)phenylphosphine, bis(2-ethylhexyl)sulfosuccinate, octylamine, dimyristoyl-L-alpha-phosphatidyl-DL-glycerol, sodium dodecylsulfonate, ascorbic acid, and sodium citrate.

14. The process as in claim 11, wherein W′ is a chemical group selected from the group consisting of R—S— and R—Se—, and wherein W is an atom selected from the group consisting of sulphur and selenium, and wherein R is selected from the group consisting of hydrogen and C₁-C₃₀ alkyl chains.

15. A film, comprising a metal nanostructure that comprises:
a nanometric metal core comprising at least one of gold, silver, and at least one of an assembly and an alloy that each comprise gold and silver; and
at least one molecule attached to at least one surface of the nanometric metal core, wherein each of the at least one molecules has the structural formula W—X—Y-Z, and wherein W includes an atom or a chemical group bound to the nanometric metal core, X includes a hydrophobic spacer, Y includes a hydrophilic spacer, and Z includes at least one of a hydrogen and a reactive group that is able to bind to at least one of a reactive substrate and a biomolecule.

16. A process for making a film, the process comprising;
suspending metal nanostructures in an aqueous medium, wherein the metal nanostructures each comprise:
a nanometric metal core comprising at least one of gold, silver, and at least one of an assembly and an alloy that each comprise gold and silver; and
at least one molecule attached to at least one surface of the nanometric metal core, wherein each of the at least one molecules has the structural formula W—X—Y-Z, and wherein W includes an atom or a chemical group bound to the nanometric metal core, X includes a hydrophobic spacer, Y includes a hydrophilic spacer, and Z includes at least one of a hydrogen and a reactive group that is able to bind to at least one of a reactive substrate and a biomolecule; and
depositing the suspended aqueous film onto a substrate.

17. A pharmaceutical composition, comprising optionally at least one pharmaceutically acceptable excipients and metal nanostructures that each comprise:
a nanometric metal core comprising at least one of gold, silver, and at least one of an assembly and an alloy that each comprise gold and silver; and
at least one molecule attached to at least one surface of the nanometric metal core, wherein each of the at least one molecules has the structural formula W—X—Y-Z, and wherein W includes an atom or a chemical group bound to the nanometric metal core, X includes a hydrophobic spacer, Y includes a hydrophilic spacer, and Z includes at least one of a hydrogen and a reactive group that is able to bind to at least one of a reactive substrate and a biomolecule.

18. The pharmaceutical composition as in claim 17, wherein the proportion of the metal nanostructures is from about 0.01 to about 99.99% by weight.

19. The pharmaceutical composition as in claim 17, wherein the proportion of the metal nanostructures in said composition is from about 0.1-10%.

20. The pharmaceutical composition as in claim 17, wherein the metal nanostructures are configured to be active under infrared irradiation.
